(19) United States
(12) Patent Application Publication
(10) Pub. No.: US 2021/0290084 A1
(43) Pub. Date: Sep. 23, 2021

(54) BLOOD FLOW SENSOR AND INFORMATION PROCESSING DEVICE

(71) Applicant: SONY CORPORATION, TOKYO (JP)

(72) Inventor: ATSUSHI ITO, TOKYO (JP)

(21) Appl. No.: 17/250,437

(22) PCT Filed: Jun. 17, 2019
(86) PCT No.: PCT/JP2019/023931

§ 371 Date: Jan. 21, 2021

(30) Foreign Application Priority Data
Jul. 31, 2018 (JP) 2018-144120

(51) Int. Cl. A61B 5/026

(52) U.S. Cl. CPC A61B 5/0261; A61B 2562/18

(57) ABSTRACT

To provide a blood flow sensor that can further reduce electrical noise. A blood flow sensor including a grounded shield portion beside a light receiver. An information processing device including one or more blood flow sensors including a grounded shield portion beside a light receiver. The grounded shield portion may be provided on an entire periphery or a partial periphery beside the light receiver. The grounded shield portion may be a grounded shield frame or a grounded shield film. A shield part of the grounded shield portion may be arranged at a position lower at least than a position of a bonding wire connecting portion.

### TECHNICAL FIELD

The present technology relates to a blood flow sensor and an information processing device including the blood flow sensor.

### BACKGROUND ART

Measurement sensors that can measure biological information such as blood flow are known. For example, blood flow can be measured by utilizing the Doppler effect of light. When blood is irradiated with light, the light is scattered by blood cells such as red blood cells. The moving speed of the blood cells is calculated from the frequency of the irradiation light and the frequency of the scattered light. Thus, there has conventionally been a technique called a laser Doppler blood flow meter for non-invasively measuring the velocity of blood flow under human skin by irradiating the skin with coherent light and then analyzing the backscattered light thereof, and various measurement devices that utilize the technique have been proposed.

For example, Patent Document 1 describes that a base includes a first accommodating recess including a first bottom surface on which a light emitting element is mounted and a second accommodating recess including a second bottom portion on which a light receiving element is mounted, and that the depth of the first accommodating recess is shallower than the depth of the second accommodating recess.

For example, Patent Document 2 proposes a laser Doppler-based blood flow measurement method in which a laser beam is incident on a measurement object and light scattered in the measurement object is received to measure the amount of blood flow of the measurement object.

Moreover, in the medical field and the like, a blood flow meter provided with a blood flow sensor is used for a technique of measuring a pulse and a blood flow velocity, which are information regarding blood flow. The blood flow meter can be worn by a subject to easily measure the pulse and blood flow velocity without causing discomfort, pain or the like to the subject. For example, Patent Document 3 proposes an information processing device capable of obtaining accurate blood flow information while reducing power consumption.

### CITATION LIST

### Patent Document

### Patent Document 1: Japanese Patent Application Laid-Open No. 2017-131286

Patent Document 2: Japanese Patent Application Laid-Open No. H8-182658

### Patent Document 3: Japanese Patent Application Laid-Open No. 2018-68428

### SUMMARY OF THE INVENTION

### Problems to be Solved by the Invention

Since the blood flow sensor is a device that amplifies and uses weak return light, it has high sensitivity and is vulnerable to electrical noise. However, it is necessary to bring the human body and the blood flow sensor close to each other although there is electrical noise from the human body. Even in a case where the blood flow sensor and the human body are provided close to each other, it is necessary to reduce the electrical noise from the human body.

Thus, it is a main object of the present technology to provide a blood flow sensor that can further reduce electrical noise.

### Solutions to Problems

The present technology can provide a blood flow sensor including a grounded shield portion beside a light receiver.

Furthermore, another aspect of the present technology can provide an information processing device including one or more blood flow sensors including a grounded shield portion beside a light receiver.

The grounded shield portion may be provided on an entire periphery or a partial periphery beside the light receiver.

The grounded shield portion may be a grounded shield frame or a grounded shield film.

A shield part of the grounded shield portion may be arranged at a position lower at least than a position of a bonding wire connecting portion.

A grounded conductor layer having an opening through which received light passes may be further arranged between the light receiver and a lid that is provided in a light receiving direction of the light receiver.

The grounded shield portion may be provided on a side surface of a second accommodating recess accommodating the light receiver.

A light source, and a base having a first accommodating recess accommodating the light source and a second accommodating recess accommodating the light receiver may be further provided.

The light receiver may be arranged at an equal interval or unequal interval in a substantially circular shape around one light source.

There may be a plurality of the light receivers, and the light receivers may be grouped and arranged as a light receiver group.

A semiconductor circuit that amplifies a light receiver output may be arranged in a region close to the light receiver group.

### Effects of the Invention

The present technology can provide a blood flow sensor that can further reduce electrical noise. Note that the effects described here are not necessarily limited, and may be any effects described in the present disclosure.

### BRIEF DESCRIPTION OF DRAWINGS

FIG. 1 is a diagram showing a blood flow sensor according to a first embodiment of the present technology.

FIG. 2 is a cross-sectional view (line A-A) of the blood flow sensor according to the first embodiment of the present technology.

FIG. 3 shows blood flow in a case where there is no side grounded conductor shield, in A, and shows blood flow in a case where there is a side grounded conductor shield, in B. The vertical axis indicates blood flow, the horizontal axis indicates time, and the left and right arrows between 10 to 30 seconds indicate a period in which measurement is performed in contact with a human body.

FIG. 4 is a diagram showing a blood flow sensor according to a second embodiment of the present technology.

FIG. 5 is a cross-sectional view (line B-B) of the blood flow sensor according to the second embodiment of the present technology.

FIG. 6 is a diagram showing a blood flow sensor according to a third embodiment of the present technology.

FIG. 7 is a cross-sectional view (line C-C) of the blood flow sensor according to the third embodiment of the present technology.

FIG. 8 is a cross-sectional view (line D-D) of the blood flow sensor according to the third embodiment of the present technology.

FIG. 9 is a diagram showing a blood flow sensor according to a fourth embodiment of the present technology.

FIG. 10 is a cross-sectional view (line E-E) of the blood flow sensor according to the fourth embodiment of the present technology.

FIG. 11 is a cross-sectional view (line F-F) of the blood flow sensor according to the fourth embodiment of the present technology.

FIG. 12 is a diagram showing modified example 1 of the blood flow sensor according to the fourth embodiment of the present technology.

FIG. 13 is a diagram showing modified example 2 of the blood flow sensor according to the fourth embodiment of the present technology.

FIG. 14 is a diagram showing modified example 3 of the blood flow sensor according to the fourth embodiment of the present technology.

FIG. 15 is a diagram showing modified example 4 of the blood flow sensor according to the fourth embodiment of the present technology.

FIG. 16 is a schematic view of a lower surface showing an example of a blood flow sensor chip provided with modified example 4 of the blood flow sensor according to the fourth embodiment of the present technology.

FIG. 17 is a schematic view of a side surface showing the example of the blood flow sensor chip provided with modified example 4 of the blood flow sensor according to the fourth embodiment of the present technology.

FIG. 18 is a schematic view of an upper surface showing the example of the blood flow sensor chip provided with modified example 4 of the blood flow sensor according to the fourth embodiment of the present technology.

FIG. 19 is a block diagram showing a functional configuration of an information processing system 1000 of the present technology.

FIG. 20 is a diagram showing an example of an embodiment of a measurement module 500 of the present technology.

FIG. 21 is a diagram showing the example of the embodiment of the measurement module 500 of the present technology.

FIG. 22 is a diagram illustrating a blood flow measurement method applied to embodiments of the present technology.

### MODE FOR CARRYING OUT THE INVENTION

Preferred modes for carrying out the present technology will be described below with reference to the drawings.

The embodiments described below show examples of representative embodiments of the present technology, and will not cause the scope of the present technology to be construed narrowly. Note that the description will be given in the following order. Note that, in the drawings, the same or equivalent elements or members are designated by the same reference numerals, and overlapping descriptions will be omitted as appropriate.

1. Blood flow sensor 1

1-1) Grounded shield portion 2

1-2) Blood flow sensor of first embodiment

1-3) Blood flow sensor of second embodiment

1-4) Blood flow sensor of third embodiment

1-5) Blood flow sensor of fourth embodiment and modified example 1 thereof

1-6) Modified examples 2 and 3 of blood flow sensor of fourth embodiment

1-7) Modified example 4 of blood flow sensor of fourth embodiment 4

2. Biometric information device 1000

### 1. Blood Flow Sensor 1

Since the blood flow sensor is a device that amplifies and uses weak return light, it has high sensitivity and is vulnerable to electrical noise. However, it is necessary to bring the human body and the blood flow sensor close to each other although there is electrical noise from the human body. Even in a case where the blood flow sensor and the human body are provided close to each other, it is necessary to reduce the electrical noise from the human body as much as possible.

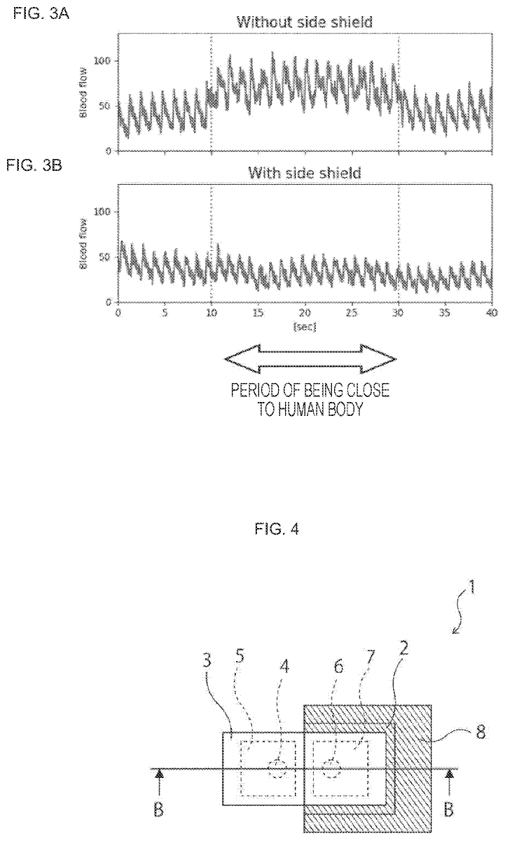

The present inventor has confirmed that, in a case where the blood flow sensor and the human body are distanced (at 1 mm or more), the electrical noise is removed by providing a conductor layer including a metal film to the blood flow sensor so as to be interposed by the human body in parallel. However, the present inventor has realized that it may be impossible to remove the electrical noise in a case where the blood flow sensor and the human body are close to each other (see FIG. 3A).

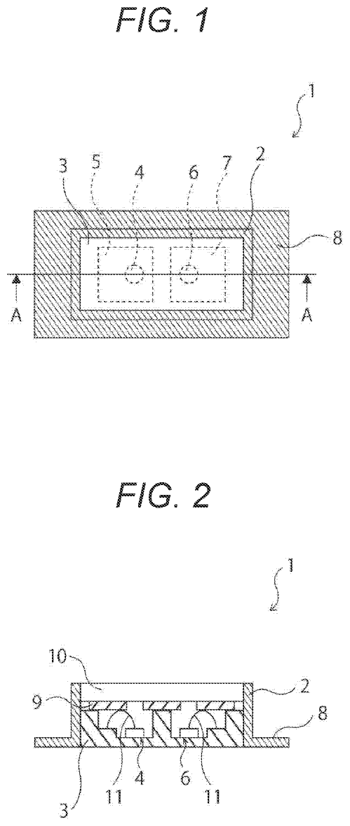

On the basis of this fact, the present inventor has made various studies on possibilities for further reducing the electrical noise. In this embodiment, a blood flow meter package is surrounded by a conductor wall including a copper tape, and installation is separately performed at a position different from the package (base) (see FIGS. 1 and 2). Note that a blood flow sensor whose side is surrounded by a conductor (copper tape) is referred to as “with side shield” (see FIGS. 1 and 2) and a blood flow sensor that is not surrounded is referred to as “without side shield” (not shown).

As [Test 1], the present inventor performed measurements using each of the above-mentioned blood flow sensors “with side shield (conductor wall)” and “without side shield (conductor wall)”. In [Test 1], measurement is performed on a fingertip through a thin transparent film (PET film 0.1 t) such that the blood flow meter and the human body do not directly contact, the human body is grounded to put the blood flow meter and the human body at the same potential for the first 10 seconds, and thereafter the human body is not grounded until 30 seconds.

As shown in FIG. 3, it can be seen that pulsation is observed and the blood flow velocity is observed while the human body is grounded. However, it can be seen that, in a case where the blood flow sensor is not provided with the conductor wall (FIG. 3A), measurement is inaccurate during a period from 10 seconds to 30 seconds because an overall offset is caused and the waveform is distorted due to the superimposition of noise. On the other hand, in a case where the conductor wall is provided (FIG. 3B), changes in blood flow velocity due to pulsation are stably observed regardless of whether the human body is grounded, and it can be seen that the noise from the human body is not electrically coupled to a photodiode (PD).

Thus, the present inventor has found that electrical noise can be reduced well by providing the grounded shield portion beside a light receiver in the blood flow sensor. That is, according to the present technology, it is possible to provide a blood flow sensor including a grounded shield portion beside a light receiver.

### 1-1) Grounded Shield Portion 2

The concept of a grounded shield portion 2 of a blood flow sensor 1 according to the present technology will be described below with reference to FIGS. 1, 4, 6, 9, 12, 13, and the like, but the present technology is not limited thereto.

The grounded shield portion 2 in the blood flow sensor of the present technology is provided beside a light receiver 6. It is preferable that the blood flow sensor of the present technology further include a base 3 having a first accommodating recess 5 accommodating a light source 4 and a second accommodating recess 7 accommodating the light receiver 6.

The grounded shield portion 2 of the present technology is preferably provided beside the base 3 housing the light receiver 6, and is further preferably provided on a side surface of the second accommodating recess 7 accommodating the light receiver 6. In a case where the grounded shield portion 2 is provided on the side surface of the second accommodating recess 7, it may be inside the second accommodating recess on the light receiver side or outside the base, but the outside of the base is preferable from the viewpoint of workability.

It is preferable that the grounded shield portion 2 in the present technology be provided on the entire periphery or a partial periphery beside the light receiver 6. Although examples of the shape, position, and the like in which the grounded shield portion 2 of the present technology is provided are shown in FIGS. 1, 4, 6, 9, 12, 13, and the like, for example, the present technology is not limited thereto.

As shown in FIGS. 1 and 4, the grounded shield portion 2 in the present technology is preferably arranged at least beside the light receiver 6, and the entire periphery or a partial periphery of the light receiver 6 is preferable.

In the case of the entire periphery, it may be the entire periphery of the base or the entire periphery of the second accommodating recess 7, but the entire periphery of the base is preferable from the viewpoint of ease of arrangement.

In the case of the partial periphery, the light receiver is not entirely surrounded. In the case of the partial periphery, it may be partially opened in the inward direction (such as the direction of the light source or the direction of the first accommodating recess). In the case of the partial periphery, it is preferable to surround the light receiver 6 such that electrical noise does not enter the light receiver 6 from beside. Example of the surrounding portions on the partial periphery include the outer peripheral portion of the side wall of the base of the second accommodating recess 7, the outer peripheral portion of at least one, two, or three or more of the wall surfaces of the side wall of the base of the second accommodating recess 7 (the front surface, rear surface, and right surface in a case where the light receiver is housed on the right side), and the like.

The shape of the grounded shield portion 2 is not particularly limited, but is preferably conformable to the side wall of the base. The grounded shield portion 2 may be configured such that a shield part on the wall surface of the grounded shield portion 2 contacts a circuit board.

Examples of the shape of the grounded shield portion 2 surrounding the side wall of the base include an L-shape, a U-shape, an arc shape, a polygonal shape, a circular shape, and the like. Examples of the polygonal shape include a triangular shape, a quadrangular shape, a rectangular shape, a pentagonal shape, a hexagonal shape, an octagonal shape, and the like.

Note that, in the case of a polygonal shape or a circumferential shape, it is used as the entire periphery, but the shape of the entire periphery may be divided into one or two or more and thereafter the divided shapes may be combined to form the shape of the entire periphery.

Furthermore, in the case of the L-shape, U-shape, arc shape, or the like, it is used as a partial periphery. Furthermore, a plurality of partial periphery shapes may be combined to surround the entire periphery or a partial periphery of the side wall of the base.

Furthermore, it is preferable that the grounded shield portion 2 further be a grounded surface 8 in order to make it easier to reduce the electrical noise, the grounded surface 8 may have a shape that contacts the surface of the circuit board, and examples of the shape include a planar shape and the like. The shape of the grounded surface 8 is preferably a shape in which the shield part on the side wall of the base extends and can be grounded with the circuit board. It is preferable that the grounded surface 8 be provided on the entire periphery or a partial periphery of the shield part on the wall surface of the grounded shield portion 2.

The material of the grounded shield portion 2 is not particularly limited as long as it is a conductive material, and examples of such metal materials include metals such as Cr, Ti, Al, Cu, Co, Ag, Au, Pd, Pt, Ru, Sn, Ta, Fe, In, Ni, and W and alloys thereof.

It is preferable that the grounded shield portion 2 be a grounded shield frame or a grounded shield film. The grounded shield frame has conductivity on the frame surface, and the molding method is not particularly limited, such as metal foil molding, metal plate molding, mold molding, injection molding, or coating molding. For example, as the molding method, the frame may be molded from a metal plate by press processing, or the surface of a resin frame may be coated with a metal film. Examples of the coating for forming the metal thin film include, but are not limited to, vapor deposition, sputtering, baking, metallization processing, and the like, and the thickness of the coating layer is 500 Å to 4000 Å, for example. The resin used for the resin frame may be either a synthetic resin or a natural resin, and may be non-conductive.

In the present technology, the grounded shield portion 2 is provided beside the light receiver 6, and it is further preferable that a grounded conductor layer 9 having an opening through which the received light passes be further arranged between the light receiver 6 and a lid 10 that is provided in the light receiving direction of the light receiver 6. In this manner, the electrical noise from the direction of the lid 10 can be further reduced. Furthermore, it is preferable that the grounded conductor layer 9 be interposed by the human body.

The grounded conductor layer 9 can be formed as a metal thin film by vapor-depositing, sputtering, baking, or the like a metal material such as the above-mentioned conductive metals or alloys thereof on the surface of the lid 10 containing a transparent ceramic material or a glass material, but there is no limitation thereto. The layer thickness of the grounded conductor layer 9 is 500 Å to 4000 Å, for example.

It is preferable that the grounded conductor layer 9 be disposed on a main surface of the lid 10, that is, the main surface on the opposite side to the main surface that the finger contacts, and is connected to the ground potential. The lid 10 may be arranged on the side opposed to the first accommodating recess 5 and the second accommodating recess 7, and the grounded conductor layer 9 may be arranged between them.

It is preferable that the grounded conductor layer 9 be provided with a first opening through which light emitted from the light source 4 passes and a second opening through which light received by the light receiver 6 passes. By providing the grounded conductor layer 9 on the main surface of the lid 10 in a region excluding the first opening and the second opening for passing light, the intrusion of electrical noise from the direction of the openings can be reduced.

The grounded conductor layer 9 can serve as a mask member provided with the first opening and the second opening such that unnecessary light is not emitted out from the first accommodating recess 5 and unnecessary light does not enter the second accommodating recess 7 from the outside.

Here, for example, Patent Document 1 discloses a structure in which a laser diode (LD) and a photodiode (PD) are provided in a package having two respective recesses and sealed with a lid containing a transparent material. Furthermore, it shows that the influence of noise generated from the human body on signals is reduced by providing a conductor layer on the back surface of the lid and grounding it.

Incidentally, since the Doppler blood flow meter thus configured is small, it is possible to wear it all the time to measure changes in blood flow velocity in daily life. However, in a case where the blood flow meter is actually used in daily life, there arises a problem that continuous changes in blood flow velocity cannot be detected because the measurement position shifts due to body movement.

One of simple measures against this can be making the positions of the blood flow meter and the human body as close as possible in order to minimize the amount of shift of the measurement position due to the body movement, for example. However, in a case where the human body and the blood flow meter are actually provided close to each other, there has been a problem that the noise generated from the human body cannot be completely prevented only by the conductor layer on the back surface of the lid as described above.

Meanwhile, for example, Patent Document 2 discloses a technique for suppressing variation in measurement results even if the measurement position shifts by using a laser Doppler blood flow meter including one light transmitting fiber and a plurality of light receiving fibers concentrically surrounding it. However, there is no description about a light-shielding structure or an electrical noise prevention structure that should be provided in a case where a large number of light receivers are used.

Furthermore, Patent Document 1 discloses a technique of distancing connection pads for wire bonding as far as possible while providing a light-shielding film in order to reduce electrical/optical noise. However, in a case where a large number of light receivers are arranged in a circle, there has been a problem that the PD and LD connection pads cannot always be sufficiently distanced. Regarding this problem, it seems possible to reduce the outer size by concentrating PDs in one place as shown in Reference 1 (Japanese Patent Application Laid-Open No. 2016-96848), for example. However, in this case, since the distance between each PD and each LD is different, the amount of light received by each PD is different and the depth of light penetration into the skin is different, resulting in acquiring information for blood vessels that are different in the depth direction, which is not desirable.

In contrast, according to the present technology, electrical noise can be further reduced by providing a grounded shield portion beside a light receiver in a blood flow sensor. Therefore, the present technology can provide a blood flow sensor that can prevent noise from a human body even if the blood flow sensor and the human body are provided close to each other. The present technology can measure a human body through a measurable, thin transparent film (such as PET) that is arranged such that the blood flow sensor and the human body do not come into direct contact with each other (such as at a space of 1 mm or less).

Furthermore, according to another aspect of the present technology, it is also possible to provide a method for reducing electrical noise in a blood flow sensor in which a conductive material is arranged beside a light receiver of the blood flow sensor. According to another aspect of the present technology, it is also possible to provide a method for reducing electrical noise in a blood flow sensor in which a grounded shield portion is provided beside a light receiver of the blood flow sensor.

Moreover, the present technology can provide a blood flow meter (information processing device) that can perform stable measurement, and it is also possible to further reduce the size of the blood flow meter. Moreover, since the electrical noise generated due to the close proximity can be prevented, the distance between the blood flow sensor and the human body can be made closer or brought into intimate contact. Moreover, in the present technology, since the blood flow sensor and the human body can be brought closer to each other, there is a possibility of providing a blood flow meter in which a measurement position does not easily shift due to body movement. Since the measurement position does not easily shift, the blood flow sensor can easily measure continuous changes in blood flow velocity in daily life.

The grounded shield portion of the present technology will be described below in more detail by showing each example of the blood flow sensor of the first to fourth embodiments, but the present technology is not limited thereto.

### 1-2) Blood Flow Sensor of First Embodiment

A first embodiment according to the present technology will be described with reference to FIGS. 1 and 2. The description of configurations overlapping with “1-1) grounded shield portion 2” described above will be omitted as appropriate.

A blood flow sensor 1 of the first embodiment of the present technology includes a grounded shield portion 2 beside a light receiver 6.

The grounded shield portion (hereinafter also referred to as “first grounded shield portion”) 2 of the first embodiment is provided on the entire periphery beside the light receiver 6. The first grounded shield portion 2 is preferably provided at least beside a base 3 accommodating the light receiver 6, and more preferably provided on a side wall on the outer periphery of the base 3.

The first grounded shield portion 2 may be a grounded shield frame or a grounded shield film. The first grounded shield portion 2 preferably has a grounded surface 8 for contacting the circuit board. The frame shape of the first grounded shield portion 2 preferably conform to the outer periphery of the side wall of the base, and is a circular shape in a case where the outer periphery of the side wall of the base has a circular shape, and is a rectangular shape in a case where the outer periphery of the side wall of the base has a rectangular shape, for example.

In the first embodiment, shielding is performed by surrounding the package of the blood flow sensor by the grounded shield portion 2 so that noise emitted from the human body is not coupled to the light receiver (PD). By adopting such a configuration, the blood flow sensor 1 of the first embodiment of the present technology can provide a blood flow sensor that can further reduce electrical noise.

The blood flow sensor 1 and a blood flow sensor package of the first embodiment of the present technology will be described below.

FIG. 1 is a view of the blood flow sensor 1 of the first embodiment as seen from a direction in which it is brought close to a human. FIG. 2 is a cross-sectional view of the first embodiment taken along line A-A.

The blood flow sensor 1 includes a base 3 that accommodates a light source 4 and a light receiver 6. The light source 4 includes at least a laser or the like, for example, and the light receiver 6 includes at least a photodiode or the like, for example.

The shape of the base 3 is not particularly limited, but is preferably a rectangular plate shape. The base 3 is formed by laminating a plurality of dielectric layers. The base 3 is provided with at least two recesses. One of the two recesses is a first accommodating recess 5 housing the light source 4, and the other is a second accommodating recess 7 accommodating the light receiver 6. The first accommodating recess 5 and the second accommodating recess 7 are provided to open a main surface provided in a direction in which it is brought close to a human.

The first accommodating recess 5 and the second accommodating recess 7 have a bottom portion for mounting the light source 4 and the light receiver 6, respectively. The distance from the mounting bottom portion to a grounded conductor layer 9 is 0.3 to 1.5 mm, for example. The bottom portion of the accommodating recess has a region in which the light source 4 or the light receiver 6 is mounted, and may have a stepped structure or a planar structure in each accommodating recess. Furthermore, the height of the bottom portion of the first accommodating recess 5 and the height of the bottom portion of the second accommodating recess 7 may be the same or different.

The size of each of the first accommodating recess 5 and the second accommodating recess 7 can be set appropriately according to the size of the light source and the light receiver to be accommodated, respectively. An example is 0.3 to 1.5 mm in the lateral direction and 0.3 to 2.0 mm in the longitudinal direction, or the like.

The opening shapes of the first accommodating recess 5 and the second accommodating recess 7 are not particularly limited, and may be, for example, a circular shape, a square shape, a rectangular shape, or the like, or may be any other shape. The size of the opening can be set appropriately according to the size of each of the light source and the light receiver to be accommodated. An example is 0.5 to 2.0 mm in the lateral direction and 0.5 to 2.5 mm in the longitudinal direction, or the like.

Furthermore, it is preferable that a first step portion having two stepped surfaces having different heights be provided on the inner surface of the first accommodating recess 5, and/or a second step portion having two stepped surfaces having different heights be provided on the inner surface of the second accommodating recess 7. Preferably, the light source is disposed on the lower step of the first step portion and a first connection pad electrically connected to the light source is disposed on the upper step, and/or the light receiver is disposed on the lower step of the second step portion and a second connection pad electrically connected to the light receiver is disposed on the upper step. The first connection pad may be disposed on the entire surface of or only a part of the upper step of the first step portion, and the second connection pad may be disposed on the entire surface of or only a part of the upper step of the second step portion. By providing the first connection pad and the second connection pad, it is possible to easily electrically connect the blood flow sensor 1, and the light source 4 and the light receiver 6 such as by bonding wires 11 and 11.

Note that the light source 4 and the light receiver 6 are electrically connected to a signal wiring conductor via the respective bonding wires 11 and 11, and the signal wiring conductor transmits an electric signal input to the light source and transmits an electric signal output from the light receiver. The signal wiring conductor includes an external connection terminal, and the external connection terminal is electrically connected to a connection terminal of an external mounting board on which the blood flow sensor is mounted by a bonding material such as solder.

The blood flow sensor 1 of the present technology further includes a lid 10 and a grounded conductor layer 9.

The lid 10 covers the first accommodating recess 5 and the second accommodating recess 7 bonded to one main surface of the base 3 like a lid. The lid 10 is a plate-shaped member containing an insulating material, and is configured to transmit light emitted from the light source 4 accommodated in the first accommodating recess 5 and to transmit light received by the light receiver 6 accommodated in the second accommodating recess 7.

Furthermore, the lid 10 is configured to pass irradiation light from the light source 4 to an object to be measured and scattered light. The lid preferably has a wavelength transmittance of 70% or more, and the lid 10 of an insulating material having a transmittance of 90% or more is more preferable from the viewpoint of detection accuracy.

Examples of the insulating material used for the lid 10 include, but are not limited to, a transparent ceramic material, a glass material, a resin material, and the like. Examples of the transparent ceramic material include sapphire and the like. Examples of the glass material include borosilicate glass, crystallized glass, quartz, soda glass, and the like. Examples of the resin material include polycarbonate resin, unsaturated polyester resin, epoxy resin, and the like.

Since the lid 10 may come into direct contact with the human body (object to be measured, such as a hand or a finger), it desirably has a predetermined strength. The strength of the lid 10 depends on the strength and plate thickness of its constituent materials. For example, in the case of a transparent ceramic material or a glass material, sufficient strength can be obtained by setting the thickness to a predetermined thickness or more (for example, a thickness of 0.05 mm to 5 mm).

### 1-3) Blood Flow Sensor of Second Embodiment

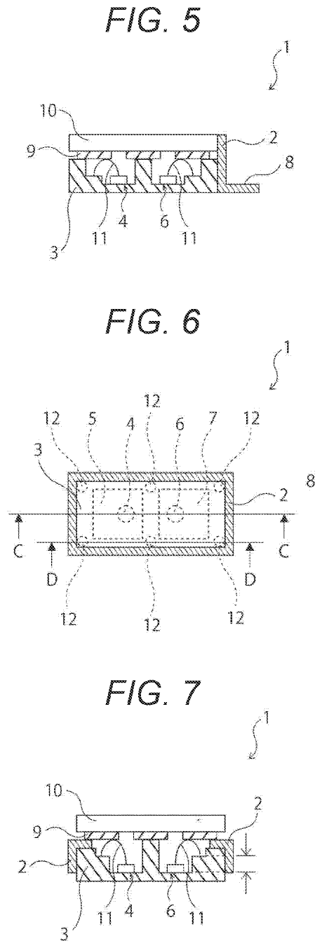

A second embodiment according to the present technology will be described with reference to FIGS. 4 and 5. The description of configurations overlapping with “1-1) grounded shield portion 2” and the first embodiment described above will be omitted as appropriate.

A blood flow sensor 1 of the second embodiment of the present technology includes a grounded shield portion 2 beside a light receiver 6.

The grounded shield portion (hereinafter also referred to as “second grounded shield portion”) 2 of the second embodiment is provided on a partial periphery beside the light receiver 6. The second grounded shield portion 2 is preferably provided at least beside a second accommodating recess 7 of a base 3 accommodating the light receiver 6, and more preferably provided on a side wall on the outer periphery of the second accommodating recess 7 of the base 3.

The second grounded shield portion 2 may be a grounded shield frame or a grounded shield film. The second grounded shield portion 2 preferably has a grounded surface 8 for contacting the circuit board. The frame shape of the second grounded shield portion 2 preferably conform to the outer periphery of the second accommodating recess 7, and is a semicircular shape in a case where the outer periphery of the second accommodating recess has a semicircular shape, and is a U-shape in a case where the outer periphery of the second accommodating recess has a U-shape, for example.

In the second embodiment, shielding is performed by providing the grounded shield portion 2 only on the side surface opposed to the receptor, so that the electrical coupling between the human body and the receptor (PD) can prevent the cause of noise superimposition. By providing the second grounded shield portion, the installation area can be reduced, so that the actual mounting size can be reduced. By adopting such a configuration as described above, the blood flow sensor 1 of the second embodiment of the present technology can provide a blood flow sensor that can further reduce electrical noise.

Note that the blood flow sensor 1 and the blood flow sensor package of the second embodiment of the present technology have configurations similar to those of the blood flow sensor 1 and the blood flow sensor package of the first embodiment of the present technology described above except for the second grounded shield portion, and the description thereof will be omitted.

### 1-4) Blood Flow Sensor of Third Embodiment

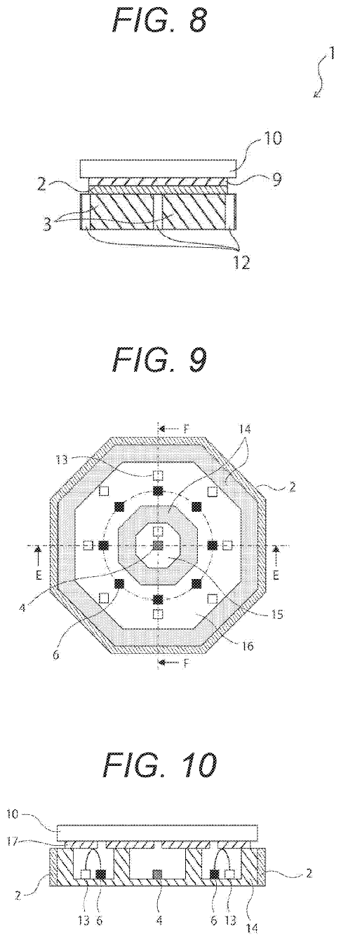

A third embodiment according to the present technology will be described with reference to FIGS. 6 and 8. The description of configurations overlapping with “1-1) grounded shield portion 2” and the first and second embodiments described above will be omitted as appropriate.

A blood flow sensor 1 of the third embodiment of the present technology includes a grounded shield portion 2 beside a light receiver 6.

The grounded shield portion (hereinafter also referred to as “third grounded shield portion”) 2 of the third embodiment is provided on the entire periphery beside the light receiver 6. The third grounded shield portion 2 is preferably provided at least beside a base 3 accommodating the light receiver 6, and more preferably provided on the entire portion or a part of a side wall on the outer periphery of the base 3.

The third grounded shield portion 2 is a grounded shield film formed by adhering a metal film of a conductor on a side surface of the base of the package. The grounded shield film can be formed by performing metallization processing, but there is no limitation thereto. The grounded shield film may cover the entire side surface of the base, or may cover only the side surface opposed to the light receiver as in the second embodiment described above.

As shown in FIG. 7, it is preferable that the third grounded shield portion 2 be formed at least to a position lower than the connecting portion where the wire bonding 11 and 11 of the light source 4 and the light receiver 6 are performed. More preferably, the third grounded shield portion 2 extends to a position lower than the lower end of the light source 4 or the light receiver 6. Further preferably, the lower end of the third grounded shield portion 2 is formed not to contact a circuit board (not shown) on which the base 3 is arranged, and it is more preferable that it be formed not to reach the lower end of the base 3 in the direction of the light receiver. Since the lower end of the third grounded shield portion 2 does not reach the lower end of the base 3, it is possible to better prevent the stirring up of solder at the time of board mounting and short circuit with the light source, the light receiver, and electrodes.

As shown in FIG. 6, for the grounding of the third grounded shield portion 2, one or more grounding peers 12 may be formed through the base so that a grounding electrode pad provided on the back surface of the base 3 is connected to the grounded shield portion 2 by using the grounding peers 12. Alternatively, for the grounding of the third grounded shield portion 2, at least a part or the entire portion of the lower end of the grounded shield film may extend to the bottom surface of the base 3 so as to be connected to a connection pad provided on the circuit board.

In the third embodiment, the grounded shield film covers the entire periphery or a partial periphery of the side surface opposed to the receptor, so that the electrical coupling between the human body and the receptor (PD) can prevent the cause of noise superimposition. By providing the grounded shield film, it is possible to perform more advanced positioning than the grounded shield frame, and workability at the time of installation becomes easier. By adopting such a configuration as described above, the blood flow sensor 1 of the third embodiment of the present technology can provide a blood flow sensor that can further reduce electrical noise.

Note that the blood flow sensor 1 and the blood flow sensor package of the third embodiment of the present technology have similar configurations to the blood flow sensor 1 and the blood flow sensor package of the first to second embodiments of the present technology described above except for the third grounded shield portion, and the description thereof will be omitted.

### 1-5) Blood Flow Sensor of Fourth Embodiment and Modified Example 1 Thereof

A blood flow sensor of a fourth embodiment according to the present technology will be described with reference to FIGS. 9 and 18. The description of configurations overlapping with “1-1) grounded shield portion 2” and the first to third embodiments described above will be omitted as appropriate.

In the blood flow sensor 1 of the fourth embodiment of the present technology, a plurality of light receivers 6 is arranged at equal intervals or unequal intervals in a substantially circular shape around one light source 4, and a grounded shield portion 2 is provided beside one or more light receivers 6. Furthermore, the light receivers 6 may be arranged in a substantially concentric circle by forming a plurality of substantial circles having different radii around the light source 4. Furthermore, the light source 4 and the light receivers 6 can be arranged concentrically in the base, and it is preferable that the light source 4 be arranged on the inner side of the base with respect to the light receiver 6.

In the blood flow sensor 1 of the fourth embodiment of the present technology, it is preferable that the grounded shield portion 2 be provided on the entire periphery or a partial periphery beside the light receiver 6. Furthermore, it is preferable that the grounded shield portion 2 be a grounded shield frame or a grounded shield film. Furthermore, it is preferable that the shield part of the grounded shield portion 2 be arranged at a position lower at least than the position of the bonding wire connecting portion.

Furthermore, in the blood flow sensor 1 of the fourth embodiment of the present technology, it is preferable that it be arranged as a light receiver group including a plurality of light receivers 6. Moreover, it is preferable that a semiconductor circuit that amplifies the light receiver output be arranged in a region close to the light receiver group.

As shown in FIG. 9, in the blood flow sensor 1 of the fourth embodiment, one light source 4 is arranged at the center of a base 14, and a plurality of light receivers 6 is arranged on a substantially circular shape around the light source 4. The base 14 preferably has a first accommodating recess 15 accommodating the light source 4 and a second accommodating recess 16 accommodating the plurality of light receivers 6. The first accommodating recess 15 is preferably formed at the center of the base 14 so as to accommodate the one light source 4. The second accommodating recess 15 is preferably formed on the outer periphery of the first accommodating recess 15 so as to accommodate the plurality of light receivers 6. Moreover, it is preferable that the grounded shield portion 2 be provided beside the second accommodating recess 15. The shape of the grounded shield portion 2 is not limited to an octagonal shape and is not particularly limited.

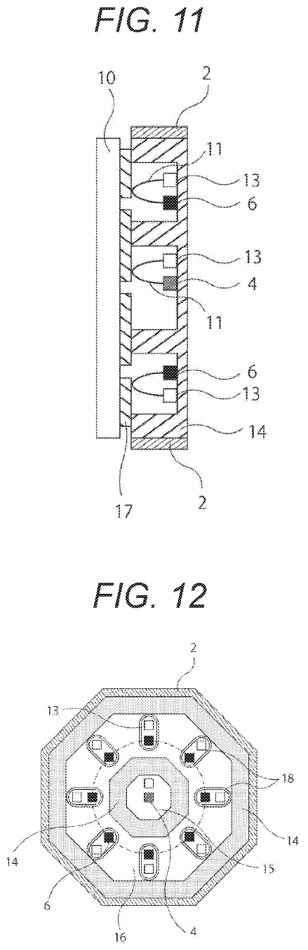

As shown in FIGS. 10 and 11, the light source 4 and the light receivers 6 are connected to connection pads 13 via respective bonding wires 11, 11, . . . . Moreover, a light-shielding film 17 having openings through which light can be output or input and a lid 10 are sequentially provided above the light source 4 and the light receivers 6. The light-shielding film 17 preferably has conductivity as described above.

Furthermore, in modified example 1 of the fourth embodiment, the grounded shield portion 2 provided beside the light receivers 6, 6, . . . arranged on the substantially circular shape may be provided as a first side shield (see FIG. 9 and the like). Furthermore, in modified example 1 of the fourth embodiment, the grounded shield portion 2 may be further provided as a second side shield 18 around each light receiver 6 (see FIG. 12 and the like). The second side shield 18 preferably surrounds the side of a group including the light receivers 6 and the connection pads 13 in the second accommodating recess 16. The second side shield 18 is preferably a grounded shield frame or a grounded shield film as described above. The second side shield 18 can be formed in a manner similar to the grounded shield portion 2 as described above. Furthermore, in a case where the second side shield 18 is provided, it is not necessary to provide the grounded shield portion 2 that is the first side shield provided beside the light receivers on the substantially circular shape.

In the blood flow sensor of the fourth embodiment, the detection accuracy of the blood flow sensor can be improved by arranging a plurality of light receivers 6 on a substantially circular shape around one light source 4 to use a large number of light receivers. The grounded shield portion of the present technology can efficiently prevent electrical noise to individual light receivers in a case where a plurality of light receivers is used, and therefore the detection accuracy of the blood flow sensor can be further improved.

### 1-6) Modified Examples 2 and 3 of Blood Flow Sensor of Fourth Embodiment

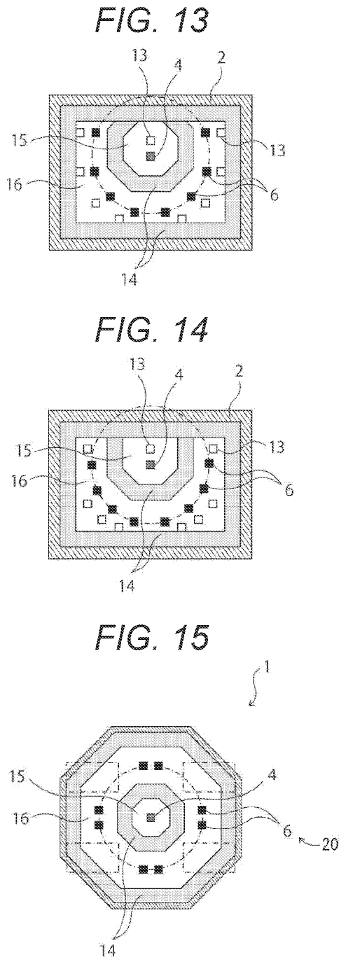

Modified examples 2 and 3 of the fourth embodiment according to the present technology will be described with reference to FIGS. 13 and 14. The description of configurations overlapping with “1-1) grounded shield portion 2” and the first to fourth embodiments described above will be omitted as appropriate.

A blood flow sensor 1 of modified example 2 or 3 of the fourth embodiment of the present technology includes a grounded shield portion 2 beside light receivers 6 on a substantially circular shape.

In the blood flow sensor of modified examples 2 and 3, it is not necessary to arrange the light source 4 at the center on the base 14, and a plurality of light receivers 6 can be arranged unevenly on the substantially circular shape of one light source 4, and a plurality of light receivers 6 can be arranged on a partial periphery, not the entire periphery, on the substantially circular shape.

Furthermore, in the blood flow sensor of modified examples 2 and 3, it is preferable that the plurality of light receivers 6, 6 . . . be unevenly arranged on a substantially circular shape around the light source 4. In providing the plurality of light receivers 6 on the substantially circular shape, the central angles of the light receiver 6 and the light receiver 6 may be the same or different, but the same central angle is preferable.

The shapes of the first accommodating recess 15 of the light source 4 and the second accommodating recess 16 of the receptor 6 are not particularly limited, the base wall is not necessarily formed around the light source 4, and it may be a polygonal shape (simple polygonal shape, convex polygonal shape, or the like), a semicircular shape, or the like as seen from above. As an example, the shapes of the first accommodating recess 15 and the second accommodating recess 16 may be a simple hexagonal shape or a simple quadrangle shape, not a regular polygonal shape, for example. For example, it is possible to reduce the outer size by setting the shape of the second accommodating recess 16 to a quadrangle shape (such as a rectangular shape or a square shape).

The grounded shield portion 2 of modified examples 2 and 3 is preferably provided on the entire periphery or a partial periphery beside each light receiver 6 in the second accommodating recess 16. Furthermore, it is preferable that the side of a group including the light receivers 6 and the connection pads 13 and the grounded shield portion 2 be grounded shield frames or grounded shield films.

In the blood flow sensor of modified examples 2 and 3, the detection accuracy of the blood flow sensor can be improved by arranging a plurality of light receivers on a substantially circular shape around one light source to use a large number of light receivers. Moreover, by making the distance between the light receivers unequal as in modified examples 2 and 3, it is possible to arrange the connection pads of the light source in a region where the distance between the light receivers is increased, for example. With such a configuration, by separating the connection pads from the light receivers, it is possible to reduce the external size while reducing electrical noise.

Also, the grounded shield portion of the present technology can appropriately follow the shape of the outer wall of the second accommodating recess due to the arrangement of the plurality of light receivers. Moreover, the grounded shield portion of the present technology can efficiently prevent electrical noise to individual light receivers in a case where a plurality of light receivers is used, and therefore the detection accuracy of the blood flow sensor can be further improved.

### 1-7) Modified Example 4 of Blood Flow Sensor of Fourth Embodiment 4

Modified example 4 of the fourth embodiment according to the present technology will be described with reference to FIGS. 15 to 18. The description of configurations overlapping with “1-1) grounded shield portion 2” and the first to fourth embodiments described above will be omitted.

A blood flow sensor 1 of modified example 4 of the fourth embodiment of the present technology includes a grounded shield portion 2 beside light receivers 6 on a substantially circular shape.

In the blood flow sensor 1 of modified example 4, a light source 4 and a plurality of light receivers 6, 6, . . . is arranged so as to surround the light source 4. In the modified example 4, the plurality of light receivers 6, 6, . . . is grouped to form light receiver groups and is arranged as light receiver groups. In this arrangement, a gap is created between the light receiver groups, and therefore electric components can be arranged in the gap portion (region portion).

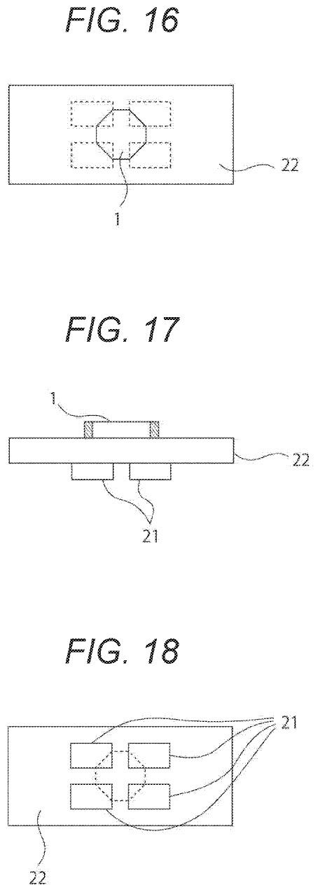

For example, in FIG. 15, eight light receivers 6 are arranged as four light receiver groups 20 by being grouped for each two of them. The light receiver groups 20 are arranged in a cross shape around the light source 4. Gap portions of the light receiver groups 20 are created in the upward, downward, leftward, and rightward diagonal directions from the center of the light source 4. Semiconductor circuit components 21 (operational amplifiers) for amplifying the output of the light receivers 6 can be provided on the back surface of a semiconductor substrate 22 between these light receiver groups (see FIGS. 16 to 18). By providing the semiconductor circuit such as the operational amplifier in this manner, the distance between the light receivers 6 and the semiconductor circuit can be minimized. By minimizing the distance, electrical noise can be reduced, and a blood flow sensor that is resistant to noise can be manufactured.

In FIG. 15, in this modified example 4, flip-chip bonding in which the light source and the light receivers are mounted on an electrode is shown, not having electrodes provided at different positions on a plate as in the case of wire bonding. However, the present technology is not limited thereto, and it may be wire bonding or the like. Furthermore, in this modified example 4, four semiconductor circuit components 21 can be provided in the upward, downward, leftward, and rightward diagonal directions of the blood flow sensor on the back surface of the surface of the semiconductor substrate 22 on which the blood flow sensor 1 is arranged.

The grounded shield portion 2 is preferably provided on the entire periphery or a partial periphery beside each light receiver 6 in the second accommodating recess 16. Furthermore, it is preferable that the side of a group including the light receivers 6 and the connection pads 13 and the grounded shield portion 2 be grounded shield frames or grounded shield films.

In the blood flow sensor of modified example 4, the detection accuracy of the blood flow sensor can be improved by arranging a plurality of light receivers on a substantially circular shape around one light source to use a large number of light receivers. Moreover, as in the modified example 4, it is also possible to provide light receiver groups and arrange semiconductor circuit components on the substrate in regions where the light receiver groups do not exist on the side opposite to the blood flow sensor. With such a configuration, it is possible to reduce the external size while reducing electrical noise.

Also, the grounded shield portion of the present technology can appropriately follow the shape of the outer wall of the second accommodating recess due to the arrangement of the plurality of light receivers. Moreover, the grounded shield portion of the present technology can efficiently prevent electrical noise to individual light receivers in a case where a plurality of light receivers is used, and therefore the detection accuracy of the blood flow sensor can be further improved.

### 2. Biometric Information Processing Device 1000

A biometric information processing device according to the present technology includes the blood flow sensor including the grounded shield portion of the present technology described above.

In embodiments of the present technology, blood flow measurement is performed in order to obtain blood flow information regarding blood flow of a subject. Specifically, the blood flow information refers to information regarding blood flow such as a pulse rate, an average blood flow velocity, a blood flow rate, and distribution in velocity of particles in blood vessels.

In embodiments of the present technology, in order to acquire the blood flow information, a part (measurement area) of a measurement subject such as a hand, arm, neck, and foot is irradiated with light, and light scattered by substances moving in blood vessels of the measurement subject and nonmoving body tissues is detected. Then, in the present embodiment, the blood flow information is acquired by processing the detected light (specifically, a detection signal).

Although embodiments of the present technology will be described by using an example of acquiring the pulse rate as a result of the blood flow measurement, the embodiments of the present technology are not limited thereto, and other blood flow information may be acquired.

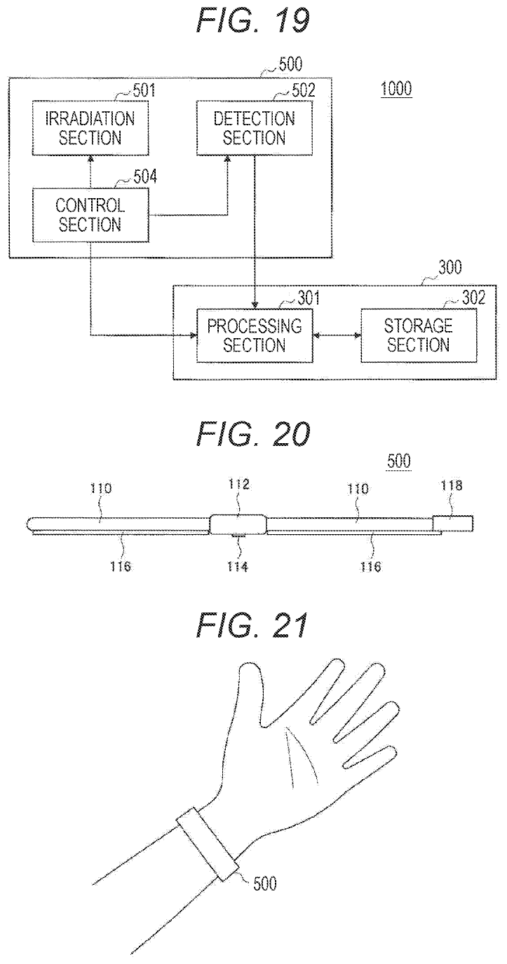

An information processing system 1000 according to the present technology includes at least the blood flow sensor 1 of the present technology. As an example of an embodiment of the information processing system 1000 of the present technology, FIG. 19 shows an information processing system mainly including a measurement module 500 including the blood flow sensor 1 of the present technology and an information processing device 300. The configuration and operation of the information processing system can use the configuration and operation in Patent Document 3, for example, but the present technology is not limited thereto.

The information processing system 1000 of the present technology may include an information display device that displays measurement results and the like to a user. The user includes the measurement subject, who is a person on which the blood flow measurement is performed, a person who uses the information processing system other than the measurement subject, or the like.

<Irradiation Section 501>

The irradiation section 501 includes a light source, and irradiates a measurement area (a part of the body) of the measurement subject with irradiation light having a predetermined wavelength from the light source. The wavelength of the irradiation light emitted by the irradiation section 501 can be selected appropriately, and it is possible to emit a wavelength of around 850 nm, for example. A small laser or the like can be used as the irradiation section 501 to emit coherent light. Also, the irradiation pattern (such as irradiation timing, irradiation duration, irradiation interval, and intensity of the irradiation light) of the irradiation section 501 can be controlled by a control section 504, which will be described later.

<Detection Section 502>

The detection section 502 detects the light scattered from the measurement area of the subject at a light receiver. The detection section 502 includes a photodiode (Photo Detector: PD), for example, converts the intensity of received light into an electric signal, and outputs it to the information processing device 300, which will be described later. Note that a charge coupled devices (CCD)-type sensor, a complementary metal oxide semiconductor (CMOS)-type sensor, or the like can also be used as the detection section 502. Furthermore, the detection section 502 may include a photodiode, an amplification circuit, a filter circuit, and an analog-digital converter, for example. Furthermore, the measurement module 500 may be provided with one or more photodiodes, sensors and the like as described above. Also, the control section 504, which will be described later, controls the detection section 502 to output the detection signal (timing or the like).

<Control Section 504>

The control section 504 controls the overall measurement in the measurement module 500, such as controlling the irradiation pattern of the irradiation section 501 and controlling the reading (sampling) timing of the detection section 502, on the basis of a predetermined synchronization signal. For example, the control section 504 controls the irradiation frequency of the irradiation section 501 and the sampling frequency of the detection section 502 synchronized with the irradiation frequency according to the operation of the information processing system 1000. Furthermore, the control section 504 may further include or access a storage section (not shown), and the storage section may store various programs, parameters, and the like for controlling the irradiation section 501 and the like. Moreover, the control section 504 may have integrated therein a mechanism for grasping other information (such as a clock mechanism; not shown) in order to output the detection signal and other information (such as time) associated with each other to the information processing device 300. For example, the control section 504 is realized by a central processing unit (CPU), a read only memory (ROM), a random access memory (RAM), or the like. Note that a part or all of the functions performed by the control section 504 may be performed by the information processing device 300, which will be described later, or an accessible information processing device (such as a sever).

<Measurement Module 500>

The measurement module 500 of the present technology includes a power source for supplying electric power to the irradiation section 501 and the like. Moreover, in addition to the irradiation section 501, detection section 502, and control section 504 described above, the measurement module 500 may include a communication section (not shown) that communicates with the information processing device 300 and the like, which will be described later. Furthermore, the measurement module 500 may include various sensors (not shown) such as a pressure sensor that detects that the measurement module is mounted to a part of the body of the measurement subject, and an acceleration sensor and a gyro sensor that detect the movement of the body.

Furthermore, the measurement module 500 can have a form as a wearable device mounted to the body of the measurement subject for use, for example. For example, the measurement module 500 may be a device that has a shape such as a wristwatch type, a ring type, a wristband type, an anklet type, a collar type, an earphone type, or the like, and can be mounted to a part of the measurement subject such as a wrist, an arm, a neck, a leg, or an ear. Furthermore, the measurement module 500 may also be a device that has a pad shape like an adhesive plaster type and can be stuck to a part of the measurement subject such as a hand, an arm, a neck, or a leg. Moreover, the measurement module 500 may have an implant-type shape to be embedded in a part of the body of the measurement subject.

An example of the specific form of the measurement module 500 according to the present embodiment will be described below with reference to FIGS. 20 and 21. For example, as shown in FIG. 20, the measurement module 500 can have a belt-like form. As shown in FIG. 20, the measurement module 500 includes a belt-like band portion 110, a control unit 112, and a measurement unit 114. The control unit 112 is a portion where the above-mentioned control section 504 is provided. Note that, in a case where the measurement module 500 and the information processing device 300, which will be described later, are an integrated device, each functional unit of the information processing device 300, which will be described later, may be provided to the control unit 112. Furthermore, the measurement unit 114 is a portion where the above-mentioned irradiation section 501 and detection section 502 are provided, and when the measurement module 500 is mounted to a part of the body of the measurement subject, it contacts or opposes to the body.

The band portion 110 is a component for fixing the measurement module 500 by being wound around a wrist of the measurement subject, for example, and includes a material such as soft silicone gel so as to form a ring shape in conformity to the shape of the wrist. That is, since the band portion 110 can have a ring shape along the wrist, the measurement module 500 can be wound around and fixed to the wrist of the measurement subject as shown in FIG. 21. Furthermore, it is preferable that the measurement module 500 be fixed over the measurement area of the measurement subject, where the measurement module 500 does not easily move during the blood flow measurement. Thus, an adhesive layer 116 that can adhere to the skin of the measurement subject may be provided at the portion of the band portion 110 that contacts the skin of the measurement subject. Further, it is preferable that the length of the circumference of the ring when the measurement module 500 is in the ring shape can be freely adjusted so that it can conform to various wrist thicknesses. Thus, a fixing portion 118 is provided at the end of the band portion 110, and the fixing portion 118 can be fixed at various positions on the band portion 110 by being overlapped with any portion on the band portion 110. In this manner, the measurement module 500 can be mounted and fixed according to the thickness of the wrist of the measurement subject.

<Information Processing Device 300>

The information processing device 300 is a device that acquires the blood flow information such as pulse by using the detection signal measured by the measurement module 500. The information processing device 300 of the present technology may include at least a processing section 301 and a storage section 302, and may further include or be able to access the measurement module 500.

The processing section 301 acquires the blood flow information by processing the detection signal obtained by the measurement module 500. The blood flow information acquired can be output to the storage section 302 or output to another device.

The storage section 302 stores programs and various data used for processing in the processing section 301, as well as the blood flow information acquired by the processing section 301 and the like. Furthermore, the storage section 302 may store parameters other than these data and the like and progresses and the like as appropriate. The processing section 301 and the like can freely access the storage section 302 to write and read data.

Note that the information processing device 300 may include a communication section (not shown) or the like for communicating with the measurement module or the like. Moreover, the information processing device 300 may include an input section (not shown) or the like that receives operations from a user who uses the information processing system 1000.

Furthermore, the information processing device 300 may be a device integrated with the above-mentioned measurement module 500, or as an information processing device 300 that may be a device separate from the measurement module 500, it may be an information processing device such as a smartphone, a tablet, or a personal computer (PC), or may be an information processing device connected to another device (such as a medical device). Further, the information processing device 300 may be an information processing device provided at a place distanced from the measurement subject, such as a sever.

<Blood Flow Measurement Method and Processing Method>

Blood flow measurement is performed in order to acquire blood flow information regarding blood flow of a measurement subject with the measurement module, information processing device, or the like including the blood flow sensor of the present technology. During measurement, a part of the measurement subject such as a hand, arm, or leg is irradiated with light, light scattered by substances moving in blood vessels of the measurement subject and nonmoving body tissues is detected, and the detected light (specifically, the detection signal) is processed.

Examples of the blood flow measurement method of the present embodiment include a laser Doppler blood flow measurement technique and a velocity distribution analysis technique using a dynamic light scattering (DLS). These can be performed by utilizing the interference phenomenon of coherent light caused by blood flow.

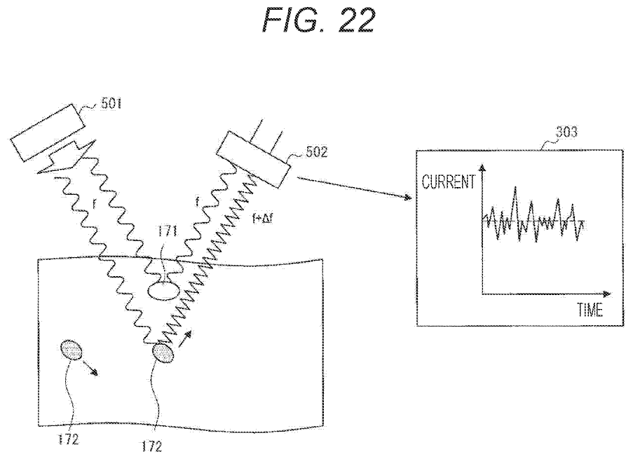

FIG. 22 schematically shows the interference phenomenon of coherent light in the blood flow, and 303 of FIG. 22 shows an example of a detected waveform obtained by the measurement.

The blood flow information measurement method in the present technology can utilize the generation of interference light that is caused by light scattered by scattering substances (mainly red blood cells) moving in blood vessels of the measurement subject when the measurement area of the measurement subject is irradiated with light emitted from the irradiation section 501 due to the Doppler effect and positional movement of the scattering substances, and is not particularly limited (for example, see Patent Document 3 or the like). The interference light is received by the detection section 502 such as a photodiode, and the blood flow information is calculated from the distribution of the Doppler shift frequency in the received interference light.

As shown in FIG. 22, in a case where light of a frequency f emitted by the irradiation section 501 and irradiating the measurement area of the measurement subject is scattered by a nonmoving tissue 171 such as the skin or the subcutaneous tissue of the measurement subject, the scattered light maintains the frequency f. On the other hand, in a case where light of the frequency f irradiating the measurement area of the measurement subject is scattered by a scattering substance (a moving particle that causes a Doppler shift of scattered light) 172 moving in a blood vessel of the measurement subject, the scattered light is frequency-shifted due to the positional movement of the scattering substance and the Doppler effect, and has a frequency of f+Δf. Examples of the scattering substance include a red blood cell, and the red blood cell is a substance having a diameter of 8 to 10 μm.

Then, since the scattered light of the frequency f scattered by the nonmoving tissue 171 and the scattered light of the frequency f+Δf scattered by the moving scattering substance 172 interfere with each other, the detection section 502 can detect interference light containing optical beat. Note that, in general, the shift frequency Δf is much smaller than the frequency f of the irradiation light.

Then, the blood flow information can be obtained by processing the interference light (detection signal) detected by the detection section 502.

In addition, the electrical noise in the blood flow sensor can be reduced by using the side shield of the present technology described above. Specifically, by arranging a conductive material beside the light receiver of the blood flow sensor as described above, electrical noise in the blood flow sensor including the grounded shield portion beside the light receiver of the blood flow sensor can be further reduced as described above. Therefore, as shown in “1. Blood flow sensor 1” above, it is possible to provide a blood flow meter (information processing device) that can perform stable measurement and reduce the size of the blood flow meter, for example.

Note that the present technology can also have the following configurations.

[1]

A blood flow sensor including a grounded shield portion beside a light receiver.

[2]

The blood flow sensor according to [1] above, in which the grounded shield portion is provided on an entire periphery or a partial periphery beside the light receiver.

[3]

The blood flow sensor according to [1] or [2] above, in which the grounded shield portion is a grounded shield frame or a grounded shield film.

[4]

The blood flow sensor according to any one of [1] to [3] above, in which a shield part of the grounded shield portion is arranged at a position lower at least than a position of a bonding wire connecting portion.

[5]

The blood flow sensor according to any one of [1] to [4] above, in which a grounded conductor layer having an opening through which received light passes is further arranged between the light receiver and a lid that is provided in a light receiving direction of the light receiver.

[6]

The blood flow sensor according to any one of [1] to [5] above, in which the grounded shield portion is provided on a side surface of a second accommodating recess accommodating the light receiver.

[7]

The blood flow sensor according to any one of [1] to [6] above, further including: a light source; and a base having a first accommodating recess accommodating the light source and the second accommodating recess accommodating the light receiver.

[8]

The blood flow sensor according to any one of [1] to [7] above, in which the light receiver is arranged at an equal interval or unequal interval in a substantially circular shape around one light source.

[9]

The blood flow sensor according to any one of [1] to [8] above, in which there is a plurality of the light receivers, and the light receivers are grouped and arranged as a light receiver group.

[10]

The blood flow sensor according to any one of [1] to [9] above, in which a semiconductor circuit that amplifies a light receiver output is arranged in a region close to the light receiver group.

[11]

An information processing device including one or more blood flow sensors including a grounded shield portion beside a light receiver. An information processing device including one or more of the blood flow sensors according to any one of [1] to

[10] above.

### REFERENCE SIGNS LIST

1 Blood flow sensor
2 Grounded shield portion
3 Base
4 Light source
5 First accommodating recess
6 Light receiver
7 Second accommodating recess
8 Grounded surface
9 Grounded conductor layer
10 Lid
11 Wire

1. A blood flow sensor comprising a grounded shield portion beside a light receiver.

2. The blood flow sensor according to claim 1, wherein the grounded shield portion is provided on an entire periphery or a partial periphery beside the light receiver.

3. The blood flow sensor according to claim 1, wherein the grounded shield portion is a grounded shield frame or a grounded shield film.

4. The blood flow sensor according to claim 1, wherein a shield part of the grounded shield portion is arranged at a position lower at least than a position of a bonding wire connecting portion.

5. The blood flow sensor according to claim 1, wherein a grounded conductor layer having an opening through which received light passes is further arranged between the light receiver and a lid that is provided in a light receiving direction of the light receiver.

6. The blood flow sensor according to claim 1, wherein the grounded shield portion is provided on a side surface of a second accommodating recess accommodating the light receiver.

7. The blood flow sensor according to claim 1, further comprising: a light source; and a base having a first accommodating recess accommodating the light source and a second accommodating recess accommodating the light receiver.

8. The blood flow sensor according to claim 1, wherein the light receiver is arranged at an equal interval or unequal interval in a substantially circular shape around one light source.

9. The blood flow sensor according to claim 8, wherein there is a plurality of the light receivers, and the light receivers are grouped and arranged as a light receiver group.

10. The blood flow sensor according to claim 9, wherein a semiconductor circuit that amplifies a light receiver output is arranged in a region close to the light receiver group.

11. An information processing device comprising one or more blood flow sensors comprising a grounded shield portion beside a light receiver.
